(19) United States
(12) Patent Application Publication
(10) Pub. No.: US 2010/0279309 A1
(43) Pub. Date: Nov. 4, 2010

(54) MICROFLUIDIC CHIPS AND SYSTEMS FOR ANALYZING PROTEIN EXPRESSION, AND METHODS OF USE THEREOF

(75) Inventor: GUODONG SUI, Boca Raton, FL (US)

Correspondence Address: Gregory A. Nelson;Novak Druce & Quigg LLP, 525 Okeechobee Blvd, Suite 1500, West Palm Beach, FL, 33401, US

(73) Assignee: Florida Atlantic University, Boca Raton, FL (US)

(21) Appl. No.: 12/743,664

(22) PCT Filed: Nov. 19, 2008
(86) PCT No.: PCT/US08/84044

§ 371 Date: May 19, 2010

Related U.S. Application Data
(60) Provisional application No. 60/989,033, filed on Nov. 19, 2007.

(51) Int. Cl. G01N 33/53; C12M 1/34

(52) U.S. Cl. 435/7.1; 435/287.2; 204/601; 204/603

(57) ABSTRACT

Microfluidic chips, systems including at least one integrated microfluidic chip operably connected to hardware (e.g., docking device, high voltage electrodes, portable fluorescence microscope, computer, etc.), and methods for analyzing peptides (e.g., toxins), proteins (e.g., cancer biomarkers, recombinant human growth hormone, recombinant human erythropoietin), and protein expression in biological samples (e.g., human serum, urine, or tissue, marine organism, seafood, etc.) have been developed. On a single microfluidic chip as described herein, several sequential processes for analyzing a protein (e.g., cell culturing, cell lysis, protein enrichment, protein labeling, and protein detection) can be performed in an automated fashion. The microfluidic chips, systems, and methods described herein provide real-time, high-throughput, highly-specific detection of proteins such as recombinant erythropoietin, recombinant human growth hormone and marine biotoxins, as well as important biomarkers in the cancer signaling pathway network for early and precise cancer diagnosis.

### FIELD OF THE INVENTION

The invention relates generally to the fields of histology, molecular biology, materials science, and microfluidics. More particularly, the invention relates to microfluidic chips, systems, and methods for analyzing toxins and proteins in a biological sample.

### BACKGROUND

At present, microscopic histology examination of patient tissue is still the standard method for cancer diagnosis. The current model of histology practice for cancer diagnosis involves microscopic examination of diseased tissue which is collected by biopsy (small operations in most cases). Based on tissue architecture patterns and the morphology of the tissue cells, as well as on the presence or absence of a very limited set of important proteins, cancer type and grade will be determined. At present it is widely accepted that any type of cancer can still be classified into different subtypes of diseases, each having their own protein expression signature. These cancer subtypes can not be distinguished from each other by traditional histology examination because they are microscopically identical in many cases. Rapid and high throughout analysis of multiple important biomarkers or proteins are greatly needed for both cancer diagnosis and molecular biology research.

Traditional western blot, enzyme-linked immunosorbant assay (ELISA), immunohistochemistry (IHC), and Immunocytochemistry (IC) are the most widely used methods for biomarker or protein analysis. These methods, however, are associated with several disadvantages. For example, they involve substantial amounts of manual work, are semi-quantitative, time consuming, and require many tissue samples. Generally, to finish one single protein analysis using one of these methods requires mg to gram amounts of sample and at least 24 hours. Typically, it takes at least two weeks to accomplish multiple protein analysis in any signaling pathway. Consequently, these methods are still research laboratory techniques rather than widely used cancer diagnosis tools.

Several automated instrumental systems have been developed. Such systems include the BioRobot LiquiChip System developed by QIAGEN (Valencia, Calif.) to perform simultaneous multiple protein analysis, the Beadlyte Cell Signaling Protein array developed by Upstate (Billerica, Mass.), the Blotless nano western system developed by CellBio Sciences (Palo Alto, Calif.), the AutoDELFIA automatic immunoassay system developed by PerkinElmer (Waltham, Mass.), and the Access® Immunoassay System developed by Beckman Coulter (Fullterton, Calif.).

However, all of these instrumentations require a substantial amount of sample preparation (e.g. cell lysis, purification) before the sample can be loaded into the instruments. Additionally, they are sample-consuming since relatively large biopsy tissue samples are needed for multiple biomarker or protein analysis. When analyzing specific proteins or peptides from a mixture of proteins from unpurified cell lysate, sensitivity and selectivity are limited. More importantly, the substantial expense of purchasing, maintaining, and training personnel to use these instrumentations prevents their further practical applications in cancer diagnosis.

In addition to cancer biomarkers, current methods of analyzing the expression of other proteins, such as erythropoietin (EPO), and peptides such as marine toxins found in seafood, have several shortcomings. There is a need for devices and methods of analyzing peptides and proteins in biological samples (e.g., from humans, seafood, marine organisms, etc.) that provide efficient, cost-effective, and accurate analysis results. Devices and methods capable of analyzing several samples in parallel in real-time for the presence of one or more peptides or proteins (e.g., marine biotoxinds, cancer biomarkers, rEPO, rhuman growth hormone) in biological samples such as urine, serum, tissue, marine organisms, and seafood are particularly desirable.

### SUMMARY

The invention relates to the development of microfluidic chips, systems including at least one integrated microfluidic chip operably connected to hardware (e.g., docking device, high voltage electrodes, portable fluorescence microscope, computer, etc.), and methods for analyzing peptides (e.g., marine toxins), proteins (e.g., cancer biomarkers, recombinant human growth hormone, recombinant human EPO), and protein expression in biological samples (e.g., serum, urine, tissue, marine organism, seafood, etc.). Microfluidic chips as described herein include column-based and capillary electrophoresis (CE)-based microfluidic chips. A typical column-based microfluidic chip for analyzing at least one peptide or protein in a biological sample includes a substrate having several components attached thereto, including: a first module for culturing and lysing cells in the sample, a second module operably connected to the first module including an affinity column and at least one detectable label for labeling the at least one peptide or protein, and a third module for immunoassay detection operably connected to the second module and including at least two columns or chambers, each of the at least two columns or chambers housing a plurality of antibodies specific to the at least one peptide or protein. A microfluidic chip can be connected to a docking device that operably connects the microfluidic chip to at least one device. Examples of devices that the microfluidic chip can be operably connected to include a cartridge for housing a buffer or reagent, a portable spectrometer, an I/O interface device, and a computer.

A typical CE-based microfluidic chip for analyzing at least one peptide or protein in a urine or serum sample includes a substrate having several components attached thereto, including: a first module for enriching the at least one peptide or protein in the sample including a support to which a plurality of antibodies specific to the at least one peptide or protein are immobilized and including at least one detectable label for labeling the enriched peptide or protein, and a second module housing a capillary electrophoresis device. In one example of operably connecting the microfluidic chip to at least one device, the microfluidic chip can be connected to a docking device. Examples of devices the microfluidic chip can be operably connected to include a cartridge for housing a buffer or reagent, a portable spectrometer, an I/O interface device, a power source for capillary electrophoresis, and a computer.

Also described herein is a system for analyzing expression of at least one peptide or protein in a biological sample (e.g., tissue, urine, serum sample, marine organism, seafood). The system includes a column-based or CE-based microfluidic chip as described herein, an I/O interface device, a spectrometer, a computer, and a docking device for operably connecting the microfluidic chip to the I/O interface, spectrometer, and computer. A system can further include a power source. A method of analyzing expression of at least one peptide or protein in a biological sample (e.g., tissue, urine, serum sample, marine organism, seafood) includes the steps of: providing a column-based or CE-based microfluidic chip as described herein; contacting the sample with the microfluidic chip under conditions where the at least one peptide or protein is enriched and labeled such that it emits UV-vis light or fluorescence; detecting UV-vis light or fluorescence emission; and correlating UV-vis light or fluorescence emission with the presence of the at least one peptide or protein in the sample.

Further described herein is a method of detecting at least one toxin in a biological sample. The method includes the steps of: providing a sample from a marine organism or a portion of seafood; providing a microfluidic chip (e.g., column-based microfluidic chip, CE-based microfluidic chip) as described herein; contacting the sample with the microfluidic chip under conditions where the at least one toxin is enriched and labeled such that it emits UV-vis light or fluorescence; detecting UV-vis light or fluorescence emission; and correlating UV-vis light or fluorescence emission with the presence of the at least one toxin in the sample. The toxin can include toxins such as microcystins, saxitoxins, Brevetoxin, Domoic Acid, Okadaic Acid, and Saxitoxin. The method can be used to screen commercial seafood for contamination with the at least one toxin. In some embodiments, a plurality of portions of seafood (e.g., portions from different organisms) can be contacted with the microfluidic chip.

A method of analyzing expression of at least one peptide or protein in a biological sample is also described herein. The method includes the steps of: providing a microfluidic chip (e.g., CE-based microfluidic chip, column-based microfluidic chip) as described herein; contacting the sample with the microfluidic chip under conditions where the at least one peptide or protein is enriched and labeled such that it emits UV-vis light or fluorescence; detecting UV-vis light or fluorescence emission; and correlating UV-vis light or fluorescence emission with the presence of the at least one peptide or protein in the sample. The biological sample can be or be isolated from human tissue, serum, urine, blood, marine organism, and seafood. In one embodiment, the biological sample is from a marine organism or seafood, and the at least one peptide or protein is a marine biotoxin.

On a single microfluidic chip as described herein, several sequential processes for analyzing a protein (e.g., cell culturing, cell lysis, protein enrichment, protein labeling, and protein detection) can be performed in an automated fashion. The microfluidic chips, systems, and methods described herein provide real-time, high-throughput, highly specific detection of peptides and proteins such as rEPO, recombinant human growth hormone (rHGH), and marine toxins that are found in seafood, as well as important biomarkers in the cancer signaling pathway network for early and precise cancer diagnosis.

The advantages of the column-based microfluidic chips described herein for use in analyzing cancer biomarker expression over traditional histology practice, for example, are the following: 1) only trace amounts of sample are needed to perform a complete histology analysis, so needle sampling will be sufficient to collect patient tissues instead of expensive surgery-based biopsy processes; 2) multiple samples from the same organ at different positions can be collected to perform parallel analyses to improve the precision and reproducibility of cancer diagnostics, especially for early stage cancer diagnosis; 3) the speed at which an analysis is performed is improved over traditional Western blot, IHC and IC analyses; 4) they can be applied to the diagnosis and study of many different types of cancers; and 5) they are typically disposable due to their low cost, providing feasibility for large-scale commercialization (e.g., hospitals).

The CE-based microfluidic chips described herein provide a number of advantages over currently available methods and devices for detecting EPO, for example. The CE-based microfluidic chips described herein provide for inexpensive and automated separation and detection of differentially glycosylated forms of EPO. They require minimal preparation of the sample (e.g., concentration and buffer exchange of urine), followed by injection of the sample into the microfluidic chip without any further user intervention. Once the sample is analyzed, the microfluidic chip can be disposed of. In some embodiments, a system for detecting EPO can include multiple microfluidic chips to analyze several samples in parallel or in a sequential manner.

Unless otherwise defined, all technical terms used herein have the same meaning as commonly understood by one of ordinary skill in the art to which this invention belongs.

By the term “toxin” is meant a toxin produced by a living organism. A “marine biotoxin” or “marine toxin” is a toxin produced by a marine organism.

As used herein, “protein” and “polypeptide” are used synonymously to mean any peptide-linked chain of amino acids, regardless of length or post-translational modification, e.g., glycosylation or phosphorylation.

As used herein, “peptide” means any of a group of organic compounds composed of two or more amino acids linked by peptide bonds.

By the term “microfluidic chip” is meant a chip containing microchannels having dimensions in the micrometer range, the microchannels capable of holding fluids. A microfluidic chip as described herein can be single layered, double layered, or have multiple layers.

By the term “module” is meant a standard unit or architecture in a microfluidic chip, which is composed of, but not limited to: microchannels, microvalves and microfilters. These units or architectures can perform microfluidic operations e.g., transporting fluids, carrying on chemical or biological reactions, etc.

The term “antibody” is meant to include polyclonal antibodies, monoclonal antibodies (mAbs), chimeric antibodies, anti-idiotypic (anti-Id) antibodies to antibodies that can be labeled in soluble or bound form, as well as fragments, regions or derivatives thereof, provided by any known technique, such as, but not limited to, enzymatic cleavage, peptide synthesis or recombinant techniques.

Although systems, devices and methods similar or equivalent to those described herein can be used in the practice or testing of the present invention, suitable systems, devices and methods are described below. All publications, patent applications, and patents mentioned herein are incorporated by reference in their entirety. In the case of conflict, the present specification, including definitions, will control. The particular embodiments discussed below are illustrative only and not intended to be limiting.

### BRIEF DESCRIPTION OF THE DRAWINGS

FIG. 1 is an illustration of a configuration of an example of a microfluidic chip system as described herein.

FIG. 2 is an illustration of the control and bottom layers of one embodiment of a microfluidic chip as described herein.

FIG. 3 is an illustration of one embodiment of a microfluidic chip for EPO detection as described herein. The device is composed of two layers; the control layer is on the top. Connection channels within the control layer are not shown and only selected valves are shown for clarity. The bottom layer is the fluidic layer where the sample concentration and electrophoresis are performed. Size of channels of the microfluidic chip: 35 μm high, 200 μm wide for most fluidic channel and 50 μm for capillary electrophoresis channel.

FIG. 4 is an illustration of one embodiment of an optical fiber-based detection system as described herein.

FIG. 5 is an illustration of a configuration of one embodiment of a reaction loop.

FIG. 6 is an illustration of a configuration of one embodiment of a microchip CE (i.e., CE-based microfluidic chip) channel.

### DETAILED DESCRIPTION

The invention provides microfluidic chips, systems, and methods for analyzing peptides, proteins, and protein expression (e.g., cancer biomarkers, recombinant human growth hormone, recombinant human erythropoietin) in biological samples (e.g., serum, urine, tissue, marine organisms, seafood, etc.). The microfluidic chips, systems, and methods for analyzing peptides, proteins, and protein expression described herein can be used to detect the presence of at least one peptide or protein in a sample, as well as to quantitate the level of the at least one peptide or protein. The below described preferred embodiments illustrate adaptations of these devices, systems and methods. Nonetheless, from the description of these embodiments, other aspects of the invention can be made and/or practiced based on the description provided below.

### Biological Methods

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). Immunology techniques are generally known in the art and are described in detail in methodology treatises such as Advances in Immunology, volume 93, ed. Frederick W. Alt, Academic Press, Burlington, Mass., 2007; Making and Using Antibodies: A Practical Handbook, eds. Gary C. Howard and Matthew R. Kaser, CRC Press, Boca Raton, Fla., 2006; Medical Immunology, 6^{th} ed., edited by Gabriel Virella, Informa Healthcare Press, London, England, 2007; and Harlow and Lane ANTIBODIES: A Laboratory Manual, Cold Spring Harbor Laboratory Press, Cold Spring Harbor, N.Y., 1988.

### Microfluidics and Capillary Electrophoresis Methods

Methods involving microfluidics devices and techniques are described herein. Such techniques are generally known in the art and are described in detail in methodology treatises such as Microfluidics for Biotechnology (Microelectromechanical systems) by Jean Berthier and Pascal Silberzan, Artech House Publishers, Norwood, Mass., 2005; Microfluidic Lab-on-a-Chip for Chemical and Biological Analysis and Discovery (Chromatographic Science) by Paul C. H. Li, CRC Press, Boca Raton, Fla., 2005; and Design Automation Methods and Tools for Microfluidics-Based Biochips, by Krishnendu Chakrabarty and Jun Zeng, Springer Press, Berlin, Germany, 2006. CE methods are also described herein. Such methods are generally known in the art and are described in detail in methodology treatises such as Microchip Capillary Electrophoresis: Methods and Protocols (Methods in Molecular Biology) by Charles Henry, 1^{st} ed., Humana Press, Totowa, N.J., 2006; and Handbook of Capillary Electrophoresis, by James P. Landers, 2^{nd} ed., CRC Press, Boca Raton, Fla., 1996.

### Microfluidic Chips

Described herein are microfluidic chips, systems including integrated microfluidic chips operably connected to hardware (e.g., I/O interface, computer, etc.), and methods of analyzing protein expression in a sample. Microfluidic devices are designed to control and manipulate microliter or nanoliter volumes of reagents in an integrated chip design. Such devices (microfluidic chips) contain functional elements that allow the precise delivery, mixing and movement of reagent solutions. They offer high surface area-to-volume ratio, fast mass and heat transfer and improved local control. Due to these advantages, microfluidic techniques offer substantially improved performance over conventional bench-top systems and are increasingly used in chemical and biomedical analyses. The advantages of these integrated microfluidic systems can overcome the existing problems and challenges in histology analysis. A column-based microfluidic chip can carry out multiple biomarker analysis based on extremely small amounts of a biological sample (e.g., patient tissue, urine, blood, marine organism, seafood, etc.). A CE-based microfluidic chip as described herein can separate and detect differentially glycosylated forms of EPO, for example, from urine samples. The integrated microfluidic chip systems described herein are automated, require no or little manual interference from laboratory personnel after initial sample preparation, and can be inexpensively pre-fabricated for disposable, single use. Although the experiments and Examples described below describe microfluidic chips for analyzing the expression of EPO, rEPO, cancer biomarkers, and marine toxins that are found in seafood, the expression of any protein can be analyzed using the microfluidic chips, systems, and methods described herein.

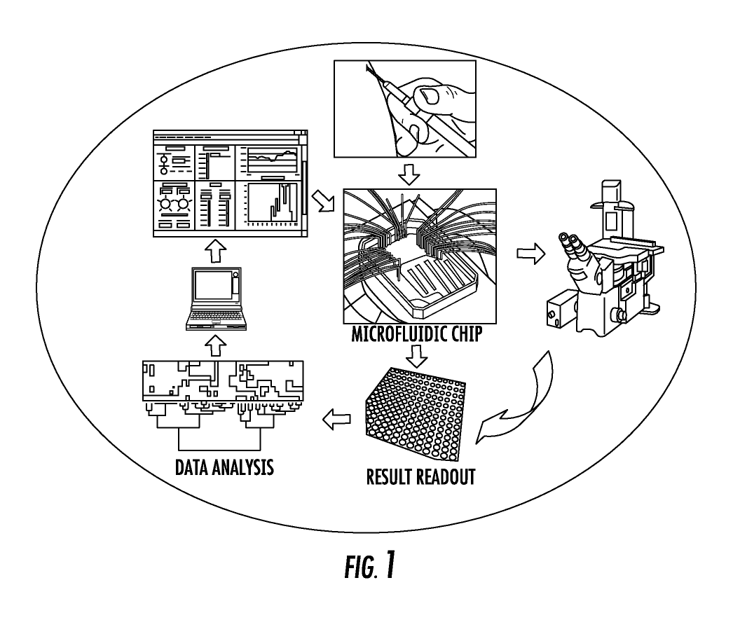

The design of the microfluidic device is based on validated microfluidic components and engineering methods. Different microfluidic components are configured onto the chip area such that they can fulfill the desired function of culturing, enriching, labeling, separating and detection of different biomarkers or proteins. The design of one example of an integrated device is shown in FIG. 1. Besides the microfluidic chip itself, the integrated device includes a docking device which connects the chip to additional hardware. This hardware includes one or more (e.g., 2, 3, 4, etc.) cartridges for buffers and reagents, a spectrometer and/or fluorometer, and a laptop computer with I/O device (e.g., DAQpad 6507 from National Instrumentation) for instrument control, data acquisition and data analysis. In one embodiment, the system operation and whole automation is programmed by LabView from National Instruments.

Generally, a microfluidic chip as described herein includes a substrate made of polydimethylsiloxane (PDMS). Microfluidic chips can be made of any suitable material, however. Examples of additional suitable materials include polycarbonate, Poly(methyl methacrylate) (PMMA) and other elastomers. Microfluidic chips are generally inexpensive to fabricate (less than $50 per chip), and are designed to be disposable for a single use. One of the advantages of a disposable chip design is that it can be manufactured under predefined quality controlled conditions, thus requiring less intervention by and training of the person performing the analysis. In addition, several antibodies recognizing different biomarker proteins can be used for the on-chip analysis system. The use of more than one antibody to detect one specific protein increases confidence of the analysis. In addition, all steps after loading the sample onto the microfluidic chip are typically performed in a completely automated manner and do not require any operator intervention.

### Integrated Microfluidic Histology Chip for Cancer Diagnosis

The invention includes integrated microfluidic histology chips for analyzing cancer biomarker expression in a tissue or serum sample and diagnosing cancer. Compared to traditional histology methods and modern instrumentation analysis for cancer diagnosis, the microfluidic chips described herein have the following unique advantages: 1) only trace amounts of samples are needed to perform a complete histology analysis, so needle sampling is sufficient to collect patient tissues instead of expensive and painful surgical biopsy processes (heretofore not feasible using current instrumentation); 2) multiple samples from the same organ at different positions can be collected to perform a parallel analysis to improve the precision and reproducibility of cancer diagnostics, especially for early-stage cancer diagnosis (heretofore not feasible using current instrumentation); 3) they can greatly improve the speed of analysis to direct the targeted therapies, compared to traditional Western blot, IHC, and IC methods; 4) they can be applied to the diagnosis and study of many different types of cancers; and 5) the analysis cost is significantly lower than current instrumentation analysis methods (making itself feasible for large scale commercialization for hospital applications).

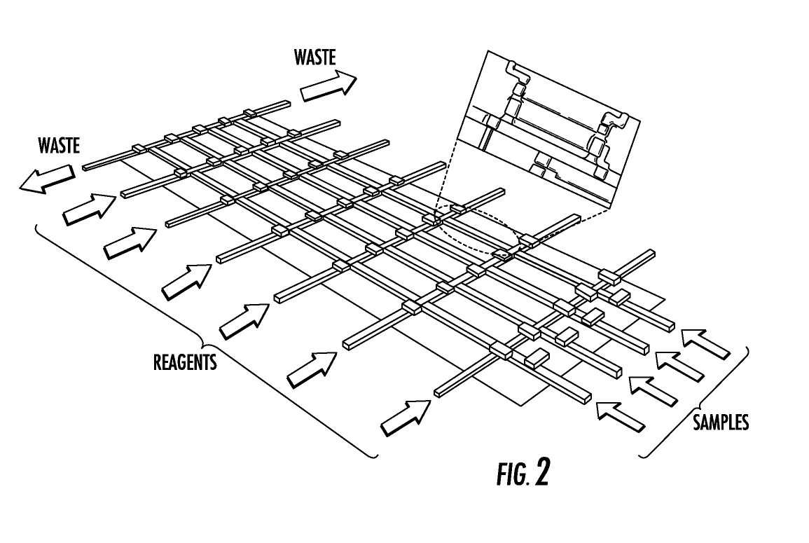

Referring to FIG. 1, a configuration of one embodiment of an integrated microfluidic chip system as described herein is illustrated. The core component of this system is the microfluidic chip. This microfluidic chip utilizes a sequence of established techniques to achieve tissue cell culturing, lysis, protein enrichment, labeling and detection. Generally, an extremely small amount of biological sample (e.g., patient tissue collected by needle sampling, seafood collected by any suitable means, etc.) containing a few hundred cells is loaded into the microfluidic chamber, where the cells are cultured and lysed to release all the biomarkers or proteins. The cell membrane debris is filtered and proteins inside the filtrate are enriched through an affinity column to increase the concentration. Then the enriched protein mixtures are labeled with a specific fluorescence dye (rhodamine or fluorescein) for fluorescence-based detection, or a dye with strong absorbance (e.g., QSY7, Molecular Probes) in the VIS region of the light spectrum for Vis absorbance detection. The enriched protein mixtures can be labeled with any suitable detectable label, however. Examples of other detectable labels that can be used including all the fluorescent or visible dye labels which are either a fluorescent molecule/visible dye molecule, or an enzyme system that generates a fluorescent product. The labeled biomarker or proteins mixtures are passed through the immunoassay detection module, a typical design for which is shown in FIG. 2. This immunoassay detection module is composed of multiple parallel orientated columns or chambers. In each immunoassay detection module, there is a blank chamber for a negative control.

On a microfluidic histology chip in which the immunoassay detection module utilizes columns, the columns are pre-loaded with microbeads (e.g., sized between approximately 5 and 20 microns) to which are adhered different trapping antibodies. The selection of the trapping antibodies used is dependent upon the protein whose expression is being analyzed. For example, if the expression of prostate specific antigen (PSA) is being analyzed, the microbeads will have adhered thereto anti-PSA antibodies specific to PSA. On a microfluidic histology chip in which the immunoassay detection module utilizes chambers (instead of columns), antibodies are directly immobilized onto the surface of the chambers. Whether columns or chambers are utilized, each has different specific antibodies (adhered directly thereto or having microbeads to which antibodies are adhered thereto) for trapping different biomarkers. Since all the proteins inside the enriched filtrate are labeled, the presence and absence of the biomarkers of interest are detected and quantitated using, for example, a fluorescence microscope as signal matrix. This signal matrix is transferred into a computer to which the microfluidic histology chip is operably connected for comparison and diagnosis. Since a computer-controlled interface can be employed, operations on microfluidic histology chips as described herein can be automated to perform multiple immunoassays simultaneously to expedite the diagnosis process.

Referring to FIG. 2, a typical microfluidic histology chip is composed of two layers: the control layer and the bottom layer. The control layer is on the top (i.e., above the bottom layer). Connection channels within the control layer are not shown and only selected valves are shown for clarity. The bottom layer is the fluidic layer where the tissue sample culturing, lysis, enrichment, labeling and immunoassay analysis are performed. In this example, the microfluidic histology chip includes 4×6 columns holding microbeads with different antibodies immobilized thereon, and is fabricated to perform multiple parallel analyses, which are color-labeled to discriminate individual columns. The dimensions of the channels in this example of a microfluidic chip are 35 μm high, and 100˜200 μm wide. The approximate cost to fabricate each microfluidic chip is approximately $50. The microfluidic chip shown in FIG. 2 is made of PDMS.

### Integrated Microfluidic Chip for Detecting rhEPO in Human Urine Samples

Also described herein is a device based on integrated microfluidics technology capable of separating and detecting differentially glycosylated forms of EPO. Based on the glycosylation pattern, non-human EPO (rEPO) can be identified from urine samples. Doping with EPO in endurance disciplines is a serious challenge to anti-doping efforts because the current EPO detection procedure is time consuming, expensive, and has been subject to challenges. Athletes can gain considerable competitive advantages from EPO use while facing a relatively low risk of being tested, convicted and punished for EPO doping. To discourage athletes from doping with EPO, it is necessary to increase their risk of being tested for EPO use at athletic events and throughout the training year. To realize this goal it is imperative to reduce the cost of testing for EPO.

A device for detecting rEPO as described herein includes a microfluidic chip. The microfluidic chip can be integrated into an instrument designed for anti-doping EPO detection. Such an instrument is largely automated and does not require manual interference from laboratory personnel after initial sample preparation. As such, it improves sample throughput in the testing laboratory and increases sample-to-sample reproducibility. The microfluidic chip can be pre-fabricated inexpensively and is generally intended for single use. A procedure for EPO detection using a microfluidic chip as described herein typically requires minimal preparation of the urine sample (i.e. concentration and buffer exchange) by a laboratory technician prior to loading the sample onto the chip, followed by injection of the sample into the microfluidic chip without any further user intervention. After completion of sample analysis, the microfluidic chip typically is disposed. If desired, the integrated system can include multiple microfluidic chips to analyze several samples in parallel or in a sequential manner. Because the underlying principle of detection and the technology of the microfluidic chip are not limited to EPO, a microfluidic chip and integrated system as described herein for detecting EPO can also be used for the detection of other doping substances, for example rHGH.

A microfluidic chip as described herein includes a substrate to which one or more structural elements (e.g., modules, hardware) are attached or operably connected. For example, a typical microfluidic chip as described herein includes structural elements necessary for: 1) enrichment of EPO from conditioned urine by affinity trapping; 2) chemical labeling of trapped EPO with chromo- or fluorophores; 3) chromatographic separation of EPO by capillary electrophoresis; 4) detection of EPO by visible or fluorescence detection; and 5) data processing and analysis. A structural element for enriching EPO from conditioned urine by affinity trapping is a module having a plurality of antibodies specific to EPO immobilized to a beaded polymeric support attached to the substrate. The module for enriching EPO can have any suitable dimensions, but is typically 200 μm×40 μm×3000 μm. Similarly, a structural element for electrophoretic separation of EPO by CE is a module having a CE device. The module for electrophoretic separation of EPO can have any suitable dimensions, but is typically 100 μm×40 μm×4000 μm. These modules are integrated into the microfluidic chip substrate. In an embodiment in which EPO is labeled with fluorophores or chromophores, the microfluidic chip is operably connected to a device for detecting the labeled EPO, such as a fluorometer.

Microfluidic chips are generally fabricated from PDMS by soft lithography. A microbead immobilized with monoclonal anti-EPO antibody is deposited into the affinity column compartment of the microchip. The derivatized EPO is released from the column and injected into an external capillary electrophoresis apparatus equipped with a UV/VIS and fluorescence detector. A micro CE-channel is integrated into the microfluidic chip. Docking sites for high voltage electrodes for CE and the optical fibers for UV/VIS and fluorescence detection are integrated into the chip.

A microfluidic chip for detecting rEPO as described herein can be tested to optimize analytical conditions of human urine samples. This can include optimization of the urine sample preparation protocol. The urine sample can be concentrated by ultrafiltration and equilibrated with a suitable buffer prior to injection into the microfluidic chip. A library of EPO profiles from a broad range of individuals is established by analyzing urine samples from individuals not exposed to recombinant EPO. Some samples are spiked with rEPO from several sources to train the software to recognize recombinant EPO from normal variability in EPO glycosylation distribution and intensity.

### Fabricating Capillary Electrophoresis Microfluidic Chips

Capillary electrophoresis microfluidic chips as described herein can be fabricated using any suitable method. For example, a microfluidic chip can be fabricated using modified multi-layer soft lithography (Sia S K, and Whitesides G M., Electrophoresis 24:3563-3576, 2003). In this method, two different molds are first fabricated by photolithographic processes to create the fluidic channels and the control channels for actuating the values located in the respective layers of the PDMS-based chemical reaction circuits. The mold used to create the fluidic channels is made by a two-step photolithographic process. In the first step, a 45-μm thick negative photoresist (SU8-2025) is spin-coated on to a silicon wafer (Silicon Quest, San Jose, USA). After UV exposure and development, a square-profiled pattern for the affinity column is obtained. In the next step, a second layer of 35-μm thick positive photoresist (AZ 50) is spin-coated on the same wafer.

Once the positive photoresist is developed, the wafer is heated above the glass transition temperature of the positive photoresist. As a result, the surface profile of the patterned positive photoresist is transformed into a round profile while the profile of the negative photoresist remains unchanged (square profile). An example of a microfluidic chip fabricated according to this method has a channel height of 35 μm and width of 200 μm and 50 μm (CE channel). Any suitable channel height and width dimensions, however, can be used. For example, the channel height can range from about 20 μm to about 50 μm. The width of a channel can range from about 20 μm to about 400 μm. The control channels mold is made by fabricating a 25 μm-thin negative photoresist (SU8-2025) pattern on a silicon wafer. In order to achieve reliable performance of each valve, the width of the control channel is set at 250 μm in sections where the valve modules are located. Before fabricating the device, both the fluidic and control molds are exposed to trimethylchlorosilane (TMSCl) vapor for 2-3 minutes. Well-mixed PDMS (GE, RTV 615 A and B in 5:1 ratio) is poured onto the fluidic mold located in a Petri dish to give a 5 mm-thick fluidic layer. In other embodiments, however, a fluidic layer between 1 mm and 10 mm can be fabricated. Another portion of PDMS (GE, RTV 615 A and B in 20:1 ratio) is spin-coated onto the control mold (1600 rpm, 60 s, ramp 15 s) to obtain the control layer. The thick fluidic layer and thin control layer are cured in an 80° C. oven for 50 minutes. After incubation, the thick fluidic layer is peeled off the mold, and holes are punched onto the fluidic layer for loading of samples and reagents as well as for aligning electrodes for microchip CE. The fluidic layer is then trimmed, cleaned and aligned onto the thin control layer. After baking at 80° C. for 60 minutes, the assembled layer is peeled off the control mold, and another set of holes is punched for access to control channels. These assembled layers are then placed on top of a glass slide that is coated (1600 rpm, 60 s, ramp 15 s) with PDMS (GE RTV 615 A and B in 20:1 ratio) that has been cured for 45 minutes in the oven. The microfluidic chip is maintained at 80° C. for 6 hours. The optical fibers (e.g., those purchased from Ocean Optics) are aligned into the fluidic layer through predefined channels in the fluidic layer before the application of the microfluidic chip.

### Antibodies

The microfluidics chips, systems, and methods described herein for analyzing protein expression in a biological sample (e.g., urine, serum, tissue, marine organism, seafood, etc.) include at least one antibody that specifically binds to the protein being analyzed. Any suitable antibodies can be used in the invention, including monoclonal and polyclonal antibodies. Methods for determining monoclonal antibody specificity and affinity by competitive inhibition can be found in Harlow, et al., Antibodies: A Laboratory Manual, Cold Spring Harbor Laboratory Press, Cold Spring Harbor, N.Y., 1988, Colligan et al., eds., Current Protocols in Immunology, Greene Publishing Assoc. and Wiley Interscience, N.Y., (1992, 1993), and Muller, Meth. Enzymol. 92:589-601, 1983, which references are entirely incorporated herein by reference.

Antibodies of the present invention can be routinely made according to methods such as, but not limited to, inoculation of an appropriate animal with the polypeptide or an antigenic fragment, in vitro stimulation of lymphocyte populations, synthetic methods, hybridomas, and/or recombinant cells expressing nucleic acid encoding such antibodies. Alternatively, antibodies for use in the invention may be commercially available.

Monoclonal antibodies that specifically bind a protein of interest (e.g., rhEPO, rHGH, cancer biomarker) may be obtained by methods known to those skilled in the art. See, for example Kohler and Milstein, Nature 256:495-497, 1975; U.S. Pat. No. 4,376,110; Ausubel et al., eds., Current Protocols in Molecular Biology, Greene Publishing Assoc. and Wiley Interscience, N.Y., (1987, 1992); Harlow and Lane ANTIBODIES: A Laboratory Manual Cold Spring Harbor Laboratory Press, Cold Spring Harbor, N.Y., 1988; Colligan et al., eds., Current Protocols in Immunology, Greene Publishing Assoc. and Wiley Interscience, N.Y., (1992, 1993), the contents of which are incorporated entirely herein by reference. Such antibodies may be of any immunoglobulin class including IgG, IgM, IgE, IgA, GILD and any subclass thereof. A hybridoma producing a monoclonal antibody of the present invention may be cultivated in vitro, in situ or in vivo.

### EXAMPLES

The present invention is further illustrated by the following specific examples. The examples are provided for illustration only and should not be construed as limiting the scope of the invention in any way.

### Example 1

### CE-Based Microfluidics Systems

CE is a microfluidic technology and is characterized by electrophoresis in a fused silica capillary column (inside diameter ˜75 micron). Recent advances in the development of microfluidics, especially the development of soft lithography, have enabled microchip-based CE instead of traditional CE (Li S F Y and Kricka L J., Clin Chem 52:37-45, 2006; Fang Z L and Fang Q., Fresenius J Anal Chem 370:978-983, 2001; Vickova et al., Journal of Liquid Chromatography & Related Technologies 29:1047-76, 2006)). The great interest in microchip CE and microfluidics is due to the potential to fabricate complex sample analyzers that can fulfill sample purification, sequential chemical reaction and sample analysis in one single microfluidic chip. Instead of bulky bench-top CE instrumentation, a portable CE instrument capable of onsite sample analysis is feasible (Jackson et al., Anal Chem 75:3643-3649, 2003; Kim et al., Microelectr Eng 78-79:563-570, 2005). In addition, significant reduction of sample and reagent costs, shorter analysis times and improved detection sensitivity compared to traditional analysis instrumentation can be achieved by microchip CE and microfluidics.

Earlier microchip CEs were mostly made from glass because of its matured fabrication method as well as its similar surface property to fused silica capillary used in traditional CE (Li S F Y and Kricka L J., Clin Chem 52:37-45, 2006). However, glass-based microchips are relatively difficult and expensive to fabricate, requiring a clean-room facility, and it is not easy to fabricate micro valves and micro pumps inside the microchip. Due to these disadvantages, PDMS has become the most important fabrication material for microchip CE (Martin et al., Anal Chem 72:3196-3202, 2000; Chen et al., Anal Chem 74:1772-1778, 2002; Vickers et al., Anal Chem 78:7446-7452, 2006; Osbourn D M and Lunte C E, Anal Chem 75:2710-2714, 2003) as well as the new generation of microfluidics (Sia S K and Whitesides G M, Electrophoresis 24:3563-3576, 2003; Ng et al., Electrophoresis et al., 23(20):3461-3473, 2002) due to its low cost, easy fabrication (can be fabricated without clean room facility), and good optical clarity. Additionally, PDMS is an excellent insulator and it prevents arcing between the high voltage electrodes in microchip CE. Its elastomeric property makes it convenient to fabricate essential microfluidic components, e.g. valves, pumps and columns within the integrated microfluidics (Lee et al., Science 310:1793-1796, 2005; Stroock et al., Science 295:647-651, 2002; Unger et al., Science 288:113-116, 2000; Groisman et al, Science 300:955-958, 2003). However, any suitable material can be used to fabricate microchip CEs, including glass, quartz and other polymers (e.g., PMMA).

With these components, PDMS-based integrated microfluidics can perform complicated chemical or biological operations, e.g. isolation, separation, active mixing, labeling and trapping processes within a single device (Lee et al., Science 310:1793-1796, 2005; Wang et al., Angew Chem Int Ed 45:5276-5281, 2006; Hansen et al., Proc Natl Acad Sci USA 99:16531-16536, 2002) in a digitally controlled manner.

### Example 2

### Detection of rEPO in Human Urine Samples by Integrated Microfluidics

EPO is a 166-amino-acid protein hormone synthesized by the kidney in response to low blood oxygenation. It stimulates the proliferation and differentiation of erythroid-progenitor cells. EPO administration leads to an increase in red blood cells and consequently increases the oxygen carrying capacity of blood. As such, it is used as a doping agent by athletes in endurance disciplines such as cycling, nordic skiing, triathlon, and similar disciplines.

EPO, which is widely available from several pharmaceutical vendors, is approved as a drug for the treatment of anemia and is frequently used during cancer chemotherapy. For these applications, EPO is produced recombinantly by genetic manipulation of mammalian cells, most frequently Chinese Hamster Ovarian (CHO) cells. Recombinant human EPO (rhEPO) produced in CHO cells is identical in its amino acids sequence to EPO produced in the human kidney (hEPO). However, the pattern of posttranslational glycosylation between EPO produced in human cells and CHO cells differs (Stubiger et al., J Sep Sci 28:1764-1778, 2005; Neususs et al., Electrophoresis 26:1442-1450, 2005). These small differences in EPO glycosylation can be used to distinguish endogenous EPO (hEPO) from exogenous EPO (rhEPO) used by athletes for doping (Stubiger et al., J Sep Sci 28:1764-1778, 2005; Neususs et al., Electrophoresis 26:1442-1450, 2005; de Frutos et al., Electrophoresis 26:1442-1450, 2005).

The analytical detection of differentially glycosylated EPO can be achieved by a number of techniques, all of which typically require a separation step followed by a detection step. For example, the EPO assay currently used by the World Anti-Doping Agency (WADA) involves separation of urinary proteins by electrophoresis and subsequent detection of EPO by immunological staining The result of this procedure is a plot showing several bands corresponding to EPOs which differ in their glycosylation. These bands are often weak and fuzzy due to limited resolution during electrophoreses and the two-fold transblotting of first EPO and then anti-EPO antibodies onto membranes.

Alternative assays have been developed and use separating techniques such as liquid chromatography mass spectrometry, capillary electrophoresis, or zone electrophoresis to separate EPO isoforms. In addition, glycosylation-specific antibodies or lectins (Nagano et al., Electrophoresis 26:1633-45, 2005) can be used to probe the pattern of EPO glycosylation. The major drawbacks of the currently used EPO anti-doping detection assay are its cost and time requirement to obtain an unambiguous result. The current protocol of electrophoretic separation, followed by blotting of the separated proteins onto a membrane support and subsequent immunological detection using antibodies and labeling reagents, requires several manual steps and limits the number of EPO tests that can be performed during a major sports event. For example, at the 2004 Olympic Summer Games, over 10,000 athletes competed and 3617 doping samples were received. Of these, 375 samples were tested for EPO and/or plasma expanders (Tsivou et al., Anal Chim Acta 555:1-13, 2006). Turn-around time for a positive EPO test was 72 hours.

The advantages of PDMS-based integrated microfluidic systems address the existing problems in current EPO detection methods and overcome the challenges in current EPO detection methods. A new microfluidic system as described herein provides an economic, portable diagnosis tool for rapid analysis of urine samples from athletes. The dramatically reduced cost and time to perform an EPO test will allow significantly more EPO tests to be conducted. The threat of increased EPO testing should result in fewer EPO doping cases.

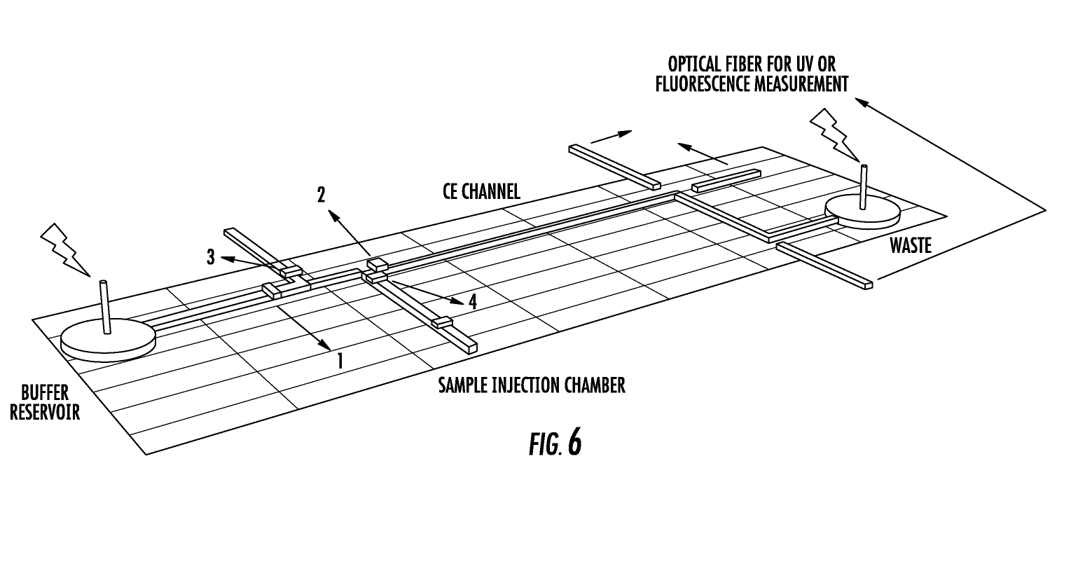

A typical design of an integrated device for detecting rEPO as described herein is shown in FIG. 6. Besides the microfluidic chip itself, the integrated device includes a docking device which connects the chip to additional hardware. This hardware includes several cartridges for buffers and reagents, the UV and/or fluorescence spectrometer components (e.g., those purchased from Ocean Optics), portable power source for microchip capillary elctrophoresis and a laptop computer with I/O device (DAQpad 6507 from National Instrumentation) for instrument control, data acquisition and data analysis. The system operation and whole automation is programmed by LabView from National Instruments (Austin, Tex.). However, any suitable programming system can be used.

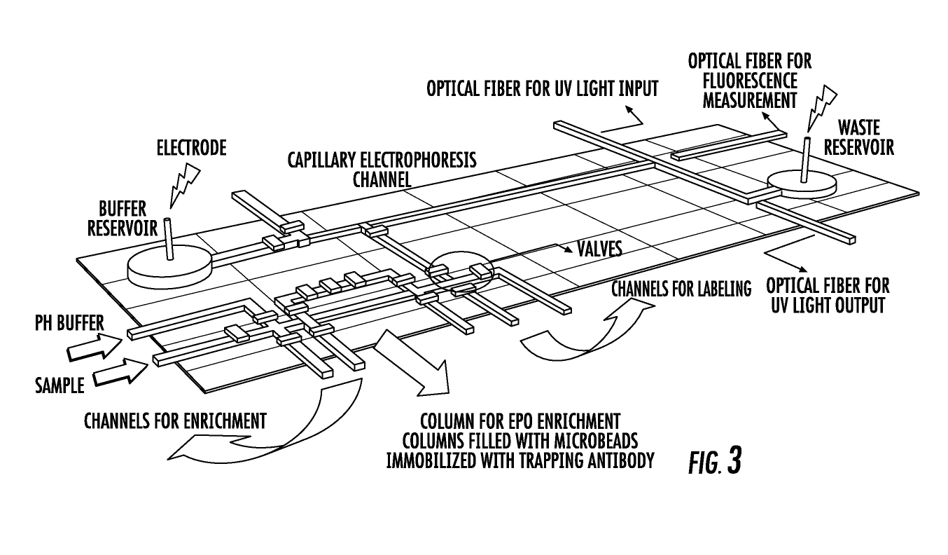

The primary component of this platform is the microfluidic chip, a detailed design of which is shown in FIG. 3. This microfluidic chip uses a sequence of well-established techniques to achieve EPO enrichment, labeling and detection. After initial urine concentration and buffer exchange, the sample is introduced into the microfluidic chip. The first step is a specific enrichment of EPO using affinity chromatography. For this purpose, specific anti-EPO antibodies are immobilized onto a beaded polymeric support trapped inside the microchip. This step also allows removal of other urine proteins. Subsequently, antibody-trapped EPO is chemically labeled with a specific fluorescence dye (rhodamine or fluorescein) for fluorescence based detection, or a dye with strong absorbance (QSY7, Molecular Probes Inc., Carlsbad, Calif.) in the UV/VIS region of the light spectrum for UV/VIS absorbance detection. After labeling, EPO is selectively released from the column and channeled into the section of the microfluidic chip where electrophoretic separation of EPO occurs. Capillary electrophoresis is used for the separation of differentially glycosylated EPO isoforms. Eluting EPO is detected at the end of the electrophoreses module by either VIS-absorbance or by fluorescence detection for increased sensitivity. The elution profile is recorded by a computer and the data is automatically analyzed.

The microfluidic chip shown in FIG. 3 is itself made of PDMS, designed to be disposable (fabrication cost less than $50 per chip) and is typically used only once. The advantage of a disposable chip design is that it can be manufactured under predefined quality controlled conditions, thus requiring less intervention by and training of the person performing the EPO analysis. In addition, several antibodies recognizing different EPO epitopes can be used for the on-chip affinity capture column. The use of more than one antibody increases confidence of the analysis. In addition, all steps after loading the sample onto the microfluidic chip are performed in a completely automated manner and do not require any operator intervention.

In one embodiment of a microfluidic chip-integrated CE system, a commonly used Spellman High Voltage DC (0˜30 KV) is utilized to drive the on-chip CE. A more economical and smaller sized EMCO DX 250 (EMCO High Voltage Corp, 1˜30 KV, 75 μA) with fixed voltage can be utilized in other embodiments (i.e., on-site sample analyzer).

For the integrated microfluidic system, valves control and direct flows inside the microfluidic channels. For the PDMS-based microfluidic chip, several different valves have been developed based on the elasticity of PDMS material. Among these valves, the pneumatic valve is chosen for a typical integrated microfluidic system due to its easy fabrication, quick response time and programmable operation. This design is composed of a double layer structure with elastomer membrane (25˜40 μm) between them. In this design, when pneumatic pressure is applied to the lower control channel, the membrane deflects and closes the upper fluidic channel.

Making multiple independently-activated valves in one chip requires independent pressure control of pressure applied to each control channel. Here, each control channel is connected with an independent electronic control valve (e.g., a device made by LEE Company). These valves can be operated at pressure up to 30 psi, with a response time less than 0.25 mS. Practically, these individual valves can be integrated into a manifold and the individual electronic control signal as well as the power supply can be provided with ribbon cable connected to a digital I/O device (e.g., the DAQPAD 6507). The simultaneous control of the multiple valves in the microfluidic chip can be managed by, for example, the DAQ6507 digital I/O interface (made by National Instrumentations), which is connected with a laptop through USB cable. The DAQPAD 6507 is capable of 96 digital I/O and can be operated by LabView programming. Only part of the 96 digital I/O is used for controlling the valves of the microfluidic chip; the other parts are utilized to control the switch of the high voltage power supply described above.

The response time of the valves from signal output from DAQPad 6507 to the complete opening or closing of the pneumatic micro valve is less than 25 mS from preliminary studies, efficient enough for standard detection on-chip operation.

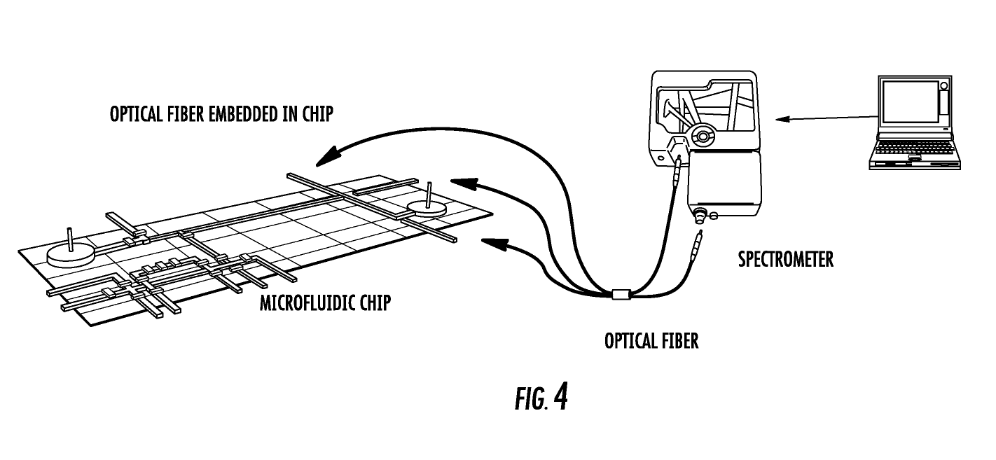

An example of a spectrometer that can be used in systems and methods as describe herein to analyze the product from the microchip CE channel is the USB 2000FLG spectrometer (Ocean Optics). The optical fiber connection between the spectrometer and microfluidic chip is shown in the following FIG. 4. The USB2000-FLG is a high sensitivity spectrofluorometer that is preconfigured for fluorescence application from 380 to 1050 nm. A proprietary thin film technology is applied in this spectrometer that makes it more sensitive compared to other spectrometers. Most importantly, it has a standard connector to assemble with the optical fibers which are embedded inside the microfluidic chip. It can acquire a full complete spectrum and data transfer within 25 mS. In addition, the USB2000 FLG is portable with dimensions of 89.1 mm×63.3 mm×34.3 mm. The signal measurement and data acquisition are controlled by the laptop through USB port. All these properties as well as a low price make it a suitable detector for the integrated microfluidic systems described herein.

On a single microfluidic chip, sample treatments including EPO enrichment, protein labeling, microchip CE, and CE detection are performed. In a microfluidic chip for detecting rEPO as described herein, the built-in affinity chromatography is responsible for the specific enrichment of the EPO from the test samples. In a typical affinity column, there are two different valves incorporated in the on-chip affinity column: the regular valve and sieve valve. A sieve valve has a square profiled fluidic channel instead of the round profiled channel in the regular valve. In both the regular valve and sieve valve, when pressure is applied in the low control channel, the elastic membrane is expanded, and the channel with the regular valve is sealed completely, while the channel with the sieve valve is be partly closed, allowing the fluid to pass through at the two edges. With this sieve valve design, solid micro beads of a certain size are trapped and the affinity column cab is packed. The dimensions of the column are 200 μm (width)×35 μm (height), suitable for trapping breads of 15 μm (diameter).

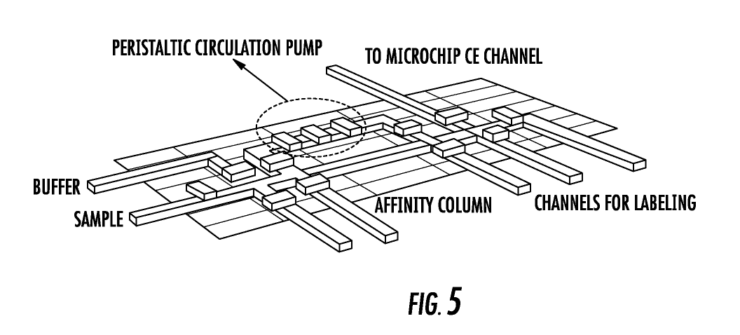

After all the EPO molecules are immobilized onto the affinity column, all other urine proteins are removed by buffer washing. Subsequently, the immobilized EPO is chemically labeled with specific fluorescent dyes (rhodamine or fluorescein) for fluorescence-based detection or a dye with strong visible absorbance (QSY 7, Molecular Probes) for visible absorbance detection. The reaction loop (FIG. 5) is responsible for the labeling of EPO molecules. The main purpose of this pump is to circulate the reagent inside the reaction loop to improve the reaction efficiency. Generally these protein labeling reactions can be completed within 5 minutes.

After the labeling, the un-reacted or excess dyes are removed by washing. The labeled EPO is then selectively released from the column by a buffer solution (pH 2) and channeled into the electrophoresis part in the microfluidic part.

The labeled EPO released from the affinity column is loaded into the sample injection chamber (purple colored chamber in FIG. 6) by adjusting the four valves (with valve 1 and 2 close and valve 3 and 4 open) adjacent to it. Then with opening valve 1 and 4 and closing valves 3 and 4, the injection chamber is isolated from the affinity column and connected with the CE channel. With applying of the high voltages to the electrodes, the labeled EPO is loaded into the CE chamber for separation.

Referring to the device shown in FIG. 6, an external capillary column is connected to the chip instead of the on-chip channel to optimize the conditions for on-chip EPO enrichment and protein labeling. Connection between the capillary column and microchip is quite tight because the hydrophobic and elasmatic property of the PMDS. In this stage, capillary column conditions reported in the literature can be adapted. With success of the on-chip EPO enrichment, labeling and external CE separation, the microfluidic chip with built-in CE channel (FIG. 6) is fabricated. In this device, the dimensions of the channel are 50 μm (width)×35 μm (height). Microfluidic chips with CE channels of different lengths (from 1 to 10 cm, snake shaped channels are used instead of linear channel for longer channels) are tested. In this stage, the optical fiber (20 μm diameter from Ocean Optics) is integrated into the microfluidic chip for transferring the signal to the external spectrometer.

In order to further improve the separation efficiency of the on-chip CE, the PDMS channel surface can be modified with polyethylene glycol (PEG) chains. PEG is a protein-resistant material and can be grafted onto the channel surface to prevent the adsorption of EPO onto the hydrophobic surface of PDMS to improve the performance of on-chip CE.

Generally, all operations from individual valve control, high voltage power supply to data collection and interpretation are controlled by a laptop computer through LabView programming. By defining the states of all on-chip valves and switches for power supply and data acquisition, as well as the period of these states, the single step operations, e.g. flushing, loading, circulation, can be defined. By defining the sequence of each step, the operations, e.g. filtration, sample enrichment, protein labeling, and sample injection into the microchip CE, can be programmed as manual, semi-automatic or totally automatic. The conditions of reagent concentration and reaction time, can be optimized. The entire reaction process can be operated in an totally automated manner.

Monodisperse microbeads (15 μm) of cross-linked polystyrene with a terminal carboxylic group are used to immobilize anti-EPO antibodies for affinity capture. The beads are activated in the microchip for covalent antibody binding with N-hydroxysuccinimide (NHS) and N-(3-dimethylaminopropyl)-N′-ethylcarbodiimide (EDC). Several monoclonal anti-EPO antibodies are commercially available (RnD Inc., StemCell Technologies Inc., Vancouver, B.C., Canada) and can be used. The specificity of the anti-EPO antibody allows the specific binding of EPO and removal of all other proteins present in the urine sample.

On-column labeling with UV/VIS, chromophores or fluorophores is performed by injecting commercial, pre-activated dyes into the microfluidic chip and guiding it into the affinity column chamber. The circulation pump on the reaction loop allows continuous passage of the reactive fluidic phase over the immobilized phase. After a set reaction time, unreacted reagent is washed out. Labeled EPO is released from the column by shifting the pH to 2, which is a standard condition for interrupting antibody-antigen interactions. The released EPO is guided into a neutralization loop and then into the loading compartment for CE. The eluted EPO can be loaded onto an external EC system and analyzed.

Conditions for optimal separation of differentially glycosylated EPO on the microchip embedded CE unit are evaluated systematically, using conditions obtained on the external EC system as initial guidance. Parameters that may be optimized are primarily CE column/channel length, voltage, and electrophoresis buffer composition.

An example of a microfluidic chip as described herein is tested and its performance is evaluated using human urine. Urine not containing exogenous EPO is obtained from volunteers. Urine samples containing recombinant rEPO are simulated by spiking human urine with commercially obtained rEPO. The rEPO shows up as additional peaks in the CE profile. A computer algorithm is trained to automatically recognize CE peaks corresponding to endogenous hEPO and to exogenous rEPO respectively. Because of some variability in the CE EPO peak profile between individuals (gender, age, race), it is important to establish a library of normal variations of EPO glycosylation to improve confidence in the recognition of rEPO used for doping.

### Example 3

### Use of Microfluidic Chips for Detecting Marine Toxins in Seafood

A low cost (disposable) microchip for detection of marine toxins in seafood based on microfluidic technology is described herein. This microchip will dramatically decrease the cost (less than $30 per chip) for assessment of seafood contaminations and water-borne pathogens. In a typical embodiment of using microfluidic chips for detecting marine toxins in seafood, existing detection methods are applied to a microfluidic chip to improve detection efficiency, shorten the detection time, and provide for on-site sample analysis.

Seafood is an important part of many Americans' diets, both nutritionally and culturally. Floridians, for example, enjoy fresh, easily obtained and inexpensive seafood whether they catch it themselves or purchase it from commercial vendors. In 2005, there were approximately 1,701,802 lbs oyster and 37,791,638 lbs finfish landing in Florida. Seafood safety is always a significant concern, especially with regard to marine toxins since most marine toxins could be destroyed by normal cooking. From 1994 to 2005, there were 58 ciguatera outbreaks (217 cases), 58 scombroid outbreaks (228 cases), 8 Neurotoxic shellfish poisonings (NSP, 17 cases) and 5 saxitoxin outbreaks (28 cases). In fact, food-borne marine toxins are not only a local problem of Florida, they are also of great concern in the northeast and the west coast of the US, as well as in Asia and Australia with different kinds of marine toxins. Generally, scombroid fish poison, ciguatera fish poison, shellfish poison (ASP, DSP, NSP, and PSP), tetrodotoxin and Gempylotoxin are the most common toxins. While several chemical methods are currently used to assess the contamination of seafood by toxic agents, the available panel of methods is far from being both complete and adequate for efficient risk management for all toxins. Monitoring of most toxins, especially the shellfish toxins, is still based on mouse bioassays, in spite of its inconsistencies, lack of specificity, and questionable ethical justification. Under these circumstances, different functional assays have been developed including cell-based assays and immunoassays as well as high performance liquid chromatography (HPLC) for some specific toxins. High costs, low-efficiency and the requirement of operation by trained professionals limits their applications, especially with regard to real-time on-site analyses.

Microfluidics is designed to control and manipulate microliter or nanoliter volumes of reagents in an integrated chip design. Such chips contain functional elements that allow the precise delivery, mixing and movement of reagent solutions. They offer high surface area-to-volume ratio, fast mass and heat transfer and improved local control. Due to these advantages, microfluidic techniques offer substantially improved performance over conventional bench-top systems and are increasingly used in bioanalysis. Herein a low cost (disposable) microchip for detection of marine toxins in seafood based on microfluidic technology is described. This microfluidic chip is capable of real-time onsite sample analysis or regular monitoring of seafood quality with minimum training requirements.

A microfluidic chip for analyzing toxins including: microcystins (LR), saxitoxins, Brevetoxin (NSP), Domoic Acid (ASP), Okadaic Acid (DSP), Saxitoxin (PSP) is designed and fabricated. The microfluidic chip is used to perform an immunoassay for detection of Ciguatera fish poisoning. A prototype of a portable instrument based on the proposed microfluidic chip for real-time and automated sample analysis is designed and tested.

Besides the core microfluidic chip itself, the portable instrument includes a docking device which connects the chip to additional hardware. This hardware includes several cartridges for buffers and reagents, the UV and/or fluorescence spectrometer components and a laptop computer with I/O device for instrument control, data acquisition and data analysis. The system operation and whole automation is programmed by LabView from National Instruments. The core component of this platform is the microfluidic chip, which is typically made of polydimethylsiloxane (PDMS), designed to be disposable (fabrication cost less than $50 per chip) and is generally used only once. Two different chips are designed for cell-based assays and immunoassays separately. A microfluidic assay for sequential ligand labeling and cell binding analysis has been designed, and a similar design is used here for a cell-based assay. Briefly, several on-chip microfluidic chambers (200 um×400 um×35 um) are pre-loaded with different cell lines for testing different toxins in one sample. The cell-lines can be pre-loaded and the whole chip is maintained at a low temperature for commercial distribution, with no need to maintain certain cell-lines culturing in the laboratory.

A microfluidic immunoassay is described herein. In a typical microfluidic immunoassay, different ligand molecules (antibodies or enzymes) are immobilized onto the inside wall of various microfluidic chambers for detecting different toxins in a food sample. In addition, all steps after loading the sample onto the microfluidic chip are performed in a completely automated manner and do not require any operator intervention. Very limited training for operating the instrument is needed. A microfluidic chip, system or method for detecting toxins in a sample as described herein generally uses a microfluidic chip as described above for detecting cancer biomarkers or EPO. In a method or system of detecting toxins in a sample (e.g., seafood, marine organism), the antibody or antibodies used bind to the toxin or toxins being tested for.

The microfluidic chips and assays described herein can be used in a variety of applications. As one example, the Environmental Protection Agency and the Department of Heath may use this low-cost marine toxin detection tool for regular measurements. As another example, shellfish and finfish farms may use this tool for more regular testing or monitoring of fish stocks. In yet a further example, the microfluidic chips and assays described herein can be used by seafood markets and seafood restaurants for food quality control. The microfluidic chips and assays described herein provide a number of advantages. They will dramatically decrease the cost of regular testing for marine toxins that contaminate seafood. The analysis efficiency will be greatly improved, as real-time monitoring is feasible instead of the traditional mouse assay or cell based assays that take several days. The low cost of the microfluidic chip (less than $30 per piece, disposable) and ease of use (little training needed instead of the traditional analysis methods handled by trained professionals) make large-scale and more frequent tests possible. The microfluidic chips and assays described herein can also be applied in the local shellfish and finfish farms for regular tests. The high technological capabilities and economic efficiency improvements provided by the microfluidic chips and assays described herein may impact nationwide or worldwide seafood industries. In addition, the large-scale fabrication of microfluidic chips may create new commercial opportunities and job markets.

Using the microfluidic platform described herein, sample labeling, incubation, cleaning and successful analysis of a cancer marker, epidermal growth factor receptor (EGFR), were all achieved within 30 minutes. The results prove that proteins (100000 copies) from 20 cells (within 0.5 microliter biological samples) are enough for a fast and clear analysis from biological samples in the system described herein.

### Other Embodiments

Any improvement may be made in part or all of the devices, systems, and method steps. All references, including publications, patent applications, and patents, cited herein are hereby incorporated by reference. The use of any and all examples, or exemplary language (e.g., “such as”) provided herein, is intended to illuminate the invention and does not pose a limitation on the scope of the invention unless otherwise claimed. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the invention. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contraindicated by context.

What is claimed is:

1. A microfluidic chip for analyzing at least one peptide or protein in a biological sample, the microfluidic chip comprising:
a. a substrate;
b. a first module for culturing and lysing cells in the sample, wherein the first module is attached to the substrate;
c. a second module operably connected to the first module comprising an affinity column and at least one detectable label for labeling the at least one peptide or protein,
wherein the second module is attached to the substrate; and
d. a third module for immunoassay detection operably connected to the second module, the third module comprising at least two columns or chambers, wherein the at least two columns or chambers each house a plurality of antibodies specific to the at least one peptide or protein.

2. The microfluidic chip of claim 1, wherein the biological sample is selected from the group consisting of: human tissue, serum, urine, blood, marine organism, and seafood.

3. The microfluidic chip of claim 1, wherein the biological sample is from a marine or seafood, and the at least one peptide or protein is a marine biotoxin.

4. A microfluidic chip for analyzing at least one peptide or protein in a biological sample, the microfluidic chip comprising:
a. a substrate;
b. a first module for enriching the at least one peptide or protein in the sample comprising a support to which a plurality of antibodies specific to the at least one peptide or protein are immobilized, and comprising at least one detectable label for labeling the enriched peptide or protein, wherein the module is attached to the substrate; and
c. a second module housing a capillary electrophoresis device, the second module operably connected to the first module,
wherein the second module is attached to the substrate.

5. The microfluidic chip of claim 4, wherein the biological sample is selected from the group consisting of: human tissue, serum, urine, blood, marine organism, and seafood.

6. The microfluidic chip of claim 4, wherein the biological sample is from seafood or a marine organism, and wherein the at least one peptide or protein is a marine biotoxin.

7. A system for analyzing expression of at least one protein in a biological sample, the system comprising;
a. a microfluidic chip according to claim 1 or claim 4;
b. an I/O interface device,
c. a spectrometer,
d. a computer; and
e. a docking device for operably connecting the microfluidic chip to the I/O interface, spectrometer, and computer.

8. The system of claim 7, further comprising a power source.

9. The system of claim 6, wherein the biological sample is selected from the group consisting of: human tissue, serum, urine, blood, marine organism, and seafood.

10. A method of analyzing expression of at least one peptide or protein in a biological sample, the method comprising the steps of:
a. providing a microfluidic chip according to claim 2 or claim 4;
b. contacting the sample with the microfluidic chip under conditions where the at least one peptide or protein is enriched and labeled such that it emits UV-vis light or fluorescence;
c. detecting UV-vis light or fluorescence emission; and
d. correlating UV-vis light or fluorescence emission with the presence of the at least one peptide or protein in the sample.

11. The method of claim 10, wherein the biological sample is selected from the group consisting of: human tissue, serum, urine, blood, marine organism, and seafood.

12. The method of claim 10, wherein the biological sample is from a marine organism or seafood, and the at least one peptide or protein is a marine biotoxin.

13. A method of detecting at least one toxin in a biological sample, the method comprising the steps of:
a. providing a sample from a marine organism or a portion of seafood;
b. providing a microfluidic chip according to claim 2 or claim 4;
c. contacting the sample with the microfluidic chip under conditions where the at least one toxin is enriched and labeled such that it emits UV-vis light or fluorescence;
d. detecting UV-vis light or fluorescence emission; and
e. correlating UV-vis light or fluorescence emission with the presence of the at least one toxin in the sample.

14. The method of claim 13, wherein the toxin is selected from the group consisting of: microcystins, saxitoxins, Brevetoxin, Domoic Acid, Okadaic Acid, and Saxitoxin.

15. The method of claim 13, wherein the method is used to screen commercial seafood for contamination with the at least one toxin.
